(19) United States
(12) Patent Application Publication
(10) Pub. No.: US 2017/0086981 A1
(43) Pub. Date: Mar. 30, 2017

(54) FEMORAL HIP STEM

(71) Applicant: Encore Medical LP dba DJO Surgical, Austin, TX (US)

(72) Inventors: Aaron BAILEY, Austin, TX (US); John GREEN, Leander, TX (US); Bryan KIRKING, Austin, TX (US); Michael SEIKEL, Austin, TX (US)

(21) Appl. No.: 15/263,002

(22) Filed: Sep. 12, 2016

Related U.S. Application Data
(63) Continuation of application No. PCT/US2015/020275, filed on Mar. 12, 2015.
(60) Provisional application No. 61/952,094, filed on Mar. 12, 2014.

(51) Int. Cl. A61F 2/36; A61F 2/30

(52) U.S. Cl. CPC A61F 2/3662; A61F 2/30767; A61F 2/367; A61F 2/3672; A61F 2/3676; A61F 2002/30369; A61F 2002/30616; A61F 2002/30769; A61F 2310/00023; A61F 2310/00029

(57) ABSTRACT

Disclosed is a tapered femoral hip stem implant for use in total hip replacement surgery. The implant is constructed using anthropomorphic data to obtain an optimal configuration for use in hip replacement surgery as well as to promote bone regrowth in the anterior proximal dimensions. In certain aspects, the implant is particularly well suited for use in an anterior approach to total hip replacement surgery due to its relatively small stem length. The implant includes a 12 degree proximal taper, which affords inherent fixation and rotational stability and a 4 degree distal taper to reduce the possibility of stress shielding.

### CROSS-REFERENCE TO RELATED APPLICATIONS

This application is a continuation of and claims priority to PCT Application PCT/US2015/020275 filed on Mar. 12, 2015, which claims priority to U.S. Provisional Application No. 61/952,094, filed on Mar. 12, 2014, the disclosure of which is hereby incorporated by reference in its entirety.

### BACKGROUND OF THE INVENTION

Field of the Invention

This application relates to a femoral hip stem that optimizes conforming proximal medial geometry with a minimally invasive insertion and subsidence resistance.

Description of the Related Technology

Hip arthroplasty or replacement refers to a surgical procedure in which the hip joint is replaced by a prosthetic implant. Total hip arthroplasty has been used since the 1960s for the treatment of destructed hip joints and replaces both the femoral component and the acetabular surface of the joint. Artificial hip joints are generally ball and socket joints, designed to match as closely as possible to the natural joint function. Generally, the artificial socket is implanted in one bone, and the artificial ball articulates in the socket. A stem structure attached to the ball is implanted in another of the patient's bones, thereby securing the ball in position. In total hip replacement surgery, a patient's natural hip is replaced by an acetabular cup component that replaces the acetabular socket, and a femoral component, or the stem-and-ball component, which replaces the femoral head.

The ball and socket join of the human hip unites the femur to the pelvis, wherein the ball-shaped head of the femur is positioned within a socket-shaped acetabulum of the pelvis. The head of the femur or ball fits into the acetabulum, forming a joint which allows for leg movement in a range of directions.

Traditionally, total hip replacement surgery involves the use of a posterior approach in which the surgeon accesses the hip joint through an incision close to the buttocks. These hip replacement techniques can require significant disturbance of the joint and connecting tissues and typically involve a relatively large incision.

During a total hip replacement, the surgeon will take a number of measurements to ensure proper prosthesis selection, limb length, and hip rotation. After making the incision, the surgeon works between the large hip muscles to gain access to the joint. The femur is pushed out of the socket, exposing the joint cavity.

In order to install the acetabular cup, the surgeon prepares the bone by reaming the acetabular socket to create a surface for accepting a cup. The cup may be held in place by bone cement or an interference or press fit, or it may have a porous outer surface suitable for bony ingrowth. The new acetabular shell is implanted securely within the prepared hemispherical socket. The plastic inner portion of the implant is placed within the metal shell and fixed into place. Then, the femur is prepared to receive the stem. The proximal end of the femur is at least partially resected to expose the central portion of the bone. Generally, at least part of the greater femoral trochanter is resected to gain access to the central portion of the femur, specifically, the medullary canal. In the central portion, a cavity is created that matches the shape of the implant stem, utilizing the existing medullary canal. The top end of the femur may be planed and smoothed. If the ball is a separate piece, the proper size is selected and attached. Finally, the ball is seated within the cup so that the joint is properly aligned, and the incision is closed.

Many efforts have been made to provide a patient with less trauma during hip arthroplasty. Utilizing an anterior approach can be advantageous. However, the anterior approach used with some traditional instruments, such as straight femoral reamers, may result in extensive trauma to the patient's tissues. Therefore, there is an unrealized need for instruments and techniques that reduce the incision size and trauma to tissues without jeopardizing preparation of the cavity of the largest appropriate size, which provide for proper sizing and alignment of the femoral component's stem, and which will improve restoration of hip function and reduce the risk of the prosthesis loosening and failing.

Moreover, there is a current unrealized need for improved devices, systems and procedures adapted for use in minimally invasive hip arthroplasty. Improved devices are desired that are adapted for introduction and operation through a smaller surgical incision than conventionally available devices. Also needed are improved devices, systems, and procedures that would minimize the damage to the flesh, muscle, and other soft tissues during insertion, operation, and withdrawal. At the same time, there is a need for improved devices, systems, and procedures that would improve sizing and aligning of the femoral components and reduce the risk of their loosening. There is a need for devices that have increased fixation both in the short and long term.

In general, devices and systems are needed that are easy to use and manufacture, minimize tissue damage, simplify surgical procedures, are versatile, allow for faster healing with fewer complications, require less post-surgical immobilization, and are less costly to produce and operate.

In general, devices and systems are needed that are easy to use and manufacture, minimize tissue damage, simplify surgical procedures, are versatile, allow for faster healing with fewer complications, require less post-surgical immobilization, and are less costly to produce and operate.

### SUMMARY

A new design for a femoral hip stem optimizes conforming proximal medial geometry with anterior approach insertion and subluxation resistance. The new design improves on existing designs by optimizing the proximal geometry. The optimization uses computer-generated models of actual femur geometry to shape the geometry of the proximal body to produce a highly conforming symmetric surface while minimizing the difficulties of inserting a highly conforming surface. More specifically, the overall length of the device is reduced to allow insertion through a smaller wound, and the conforming geometry is tapered such natural variations in bone geometry won't cause high stress contacts with the highly conforming surfaces.

In one aspect, a tapered femoral prosthetic implant for use in total hip replacement is provided. The implant advantageously has a first taper portion configured to receive and attach to a femoral head element; and a stem portion. The stem portion may include a proximal stem region and a distal stem region, wherein the proximal stem region includes a 12 degree anterior proximal taper; and wherein the distal stem region has a 4 degree distal taper. The implant further includes an exterior surface, which may include a porous coating.

The stem portion may include a distal tip. In certain aspects, the distal tip includes a beveled configuration. The distal tip may facilitate insertion of said implant during implantation.

An improved method for treating osteoarthritis and fractures of a hip of a patient through replacement of the hip with a femoral hip stem is likewise described. The femoral hip stem facilitates insertion of the stem and visual confirmation of fit of the stem while also reducing trauma to tissue and femur of the patient. The method may include providing the femoral hip stem wherein as dimensions of the femur increase in both an anterior and posterior dimension from a diaphysial region of the femur to a proximal metaphysial region of the femur, dimensions of a proximal end of the stem also increase in both an anterior and posterior dimension relative to a distal end of the stem to provide a stem shape congruent to the femur of the patient and optimize proximal geometry of the stem for insertion of the stem. The method may further include making an incision in the patient and removing a portion of the femur to create a cavity. Advantageously, the stem is positioned in the cavity such that the proximal geometry of the stem is visible at a resection plane of the stem to allow visual feedback of the fit of the stem.

Advantageously, the stem shape is most congruent to the femur of the patient at the resection plane of the stem. The distal end of the stem may include a beveled tip to protect a lateral cortical bone of the patient. The proximal end of the stem may include a plurality of contact points at the resection plane to allow visual feedback that the plurality of contact points are congruent with the patient anatomy. The dimensions of stem decrease in both a medial and a lateral dimension from the proximal end of the stem to the distal end of the stem. The dimension of the stem decreases in a distal dimension from the proximal end of the stem to the distal end of the stem. Optionally, the incision is between about 5 and 10 cm in length.

The method may be employed utilizing an incision that is either a posterior, anterior, or lateral incision.

In yet another aspect, an improved method for treating osteoarthritis and fractures of a hip of a patient through replacement of the hip with a femoral hip stem is provided. The method minimizes subsidence risk, reduces trauma to tissue and femur of the patient, and improves recovery time, the method includes providing the femoral hip stem wherein the stem has both a distal taper and a proximal taper. The distal taper is configured to reduce the incidence of stress shielding and the proximal taper increases fixation and rotational stability through increased surface area contact with cortical bone structures. The improved method may further include making an incision in the patient; removing a portion of the femur to create a cavity; and setting the stem in the cavity. Preferably, the distal and proximal taper limit micromotion between the stem and the femur in both a medial and lateral dimension. The incision may be an anterior incision that reduces soft tissue damage. Optionally, a surface of the stem may be coated with a porous coating to facilitate rapid bone in-growth. In still other aspects, the distal taper is a 4 degree distal taper. The proximal taper may include a 12 degree proximal taper.

A kit for an improved treatment of osteoarthritis through replacement of a hip is likewise contemplated. The kit may include a femoral hip stem wherein as dimensions of a femur increase in both an anterior and posterior dimension from a diaphysial region of the femur to a proximal metaphysial region of the femur, dimensions of a proximal end of the stem also increase in both an anterior and posterior dimension relative to a distal end of the stem to provide a stem shape congruent to the femur and optimize proximal geometry of the stem for insertion of the stem; and instructions for positioning and setting the femoral hip stem in a cavity of the femur through an anterior incision. In certain aspects, the femoral hip stem includes a porous coating. The instructions may include a guide to visually confirm correct positioning and setting of the femoral hip stem.

### BRIEF DESCRIPTION OF THE DRAWINGS

FIGS. 1A-1D are various views of a prosthetic femoral implant. FIG. 1A is a side view of a prosthetic femoral implant. FIG. 1B is an isometric view of an implant. FIG. 1C is the same implant at perspective view and FIG. 1D is a top view of an implant.

FIG. 2 is a lateral view of a prosthetic femoral implant

FIGS. 3A-3C are perspective views of ADaMs femur models.

FIG. 4 is a model of the proximal region of a femur and illustrates the location of cross sections where implant and femur fit were evaluated.

FIGS. 5A and 5B illustrate side and perspective views, respectively, of the size progression of an implant.

FIG. 6 is a side view of a femoral implant illustrating the length (L) of the stem portion.

FIG. 7 is a side view of a femoral implant illustrating the sizing and dimensions of the stem implant.

FIG. 8 is a cross section of a femoral implant of FIG. 7, taken along the C-C axis.

FIG. 9A is a cross-sectional view of a femoral implant in situ with a tapered design.

FIG. 9B is a cross-sectional view of a prior art femoral implant in situ.

### DETAILED DESCRIPTION OF CERTAIN INVENTIVE EMBODIMENTS

After reading this description, it will become apparent to one skilled in the art how to implement the invention in various alternative embodiments and alternative applications. However, all the various embodiments of the present invention will not be described herein. It is understood that the embodiments presented here are presented by way of an example only, and not limitation. As such, this detailed description of various alternative embodiments should not be construed to limit the scope or breadth of the invention as set forth below.

Embodiments of this application relate to the optimization of proximal fit in a hip stem implant having a shorter stem. An anterior approach during total hip surgery can be advantageous for its less invasive technique as compared to a more traditional posterior approach. The anterior approach for total hip replacement is a tissue-sparing alternative to traditional hip replacement surgery that provides the potential for less pain, faster recovery, reduced hospital stays, and improved mobility because the muscle tissues are spared during the surgical procedure. The technique allows the surgeon to work between muscles and tissues without detaching the muscles from either the hip or thighbones, thereby sparing the tissue from trauma. Keeping the muscles intact may also help to prevent dislocations. With an anterior approach, the surgeon can employ a relatively small incision on the anterior of the hip as opposed to the side or back. Accordingly, with a front incision, a patient can avoid the pain of sitting on the incision site.

Minimally invasive arthroplasty techniques may further benefit a patient by providing reducing soft-tissue exposure and minimizing trauma to the muscle and ligament mechanisms. However, the less invasive technique necessarily results in a surgical window with restrictive geometry which complicates insertion and placement of longer femoral components. Disclosed herein is a femoral hip stem with a shortened overall length to facilitate insertion with an anterior approach and an anatomically congruent, tapered profile to maintain stability and prevent subsidence. Features of the invention include, without limitation, a device which is inserted with greater ease due to its stem size, increased fixation stability afforded by the proximal body conformity as well as optional coating as will be described in greater detail below, a reduced risk of initial subsidence, and a lower fracture risk as compared to highly conforming devices without regard to anatomical variation. Disclosed herein is a tapered hip system which provides increased stability and reduced trauma in less invasive hip approaches. The invention is based, in part, on the surprising discovery that the disclosed tapered implant performs so well for both the surgeon and the patient receiving the implant. As will be described in greater detail below, the implant, designed using anthropomorphic data and a unique algorithm to optimize fit and performance of the device, is a boon to femoral hip prostheses.

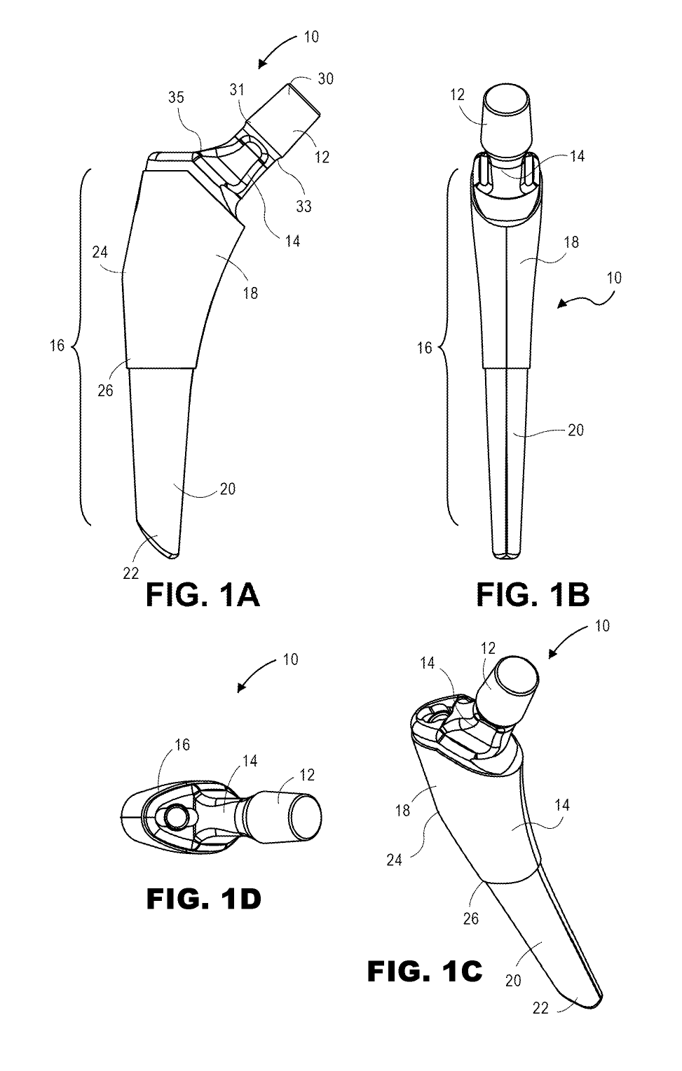

Features of the femoral hip system include its tapered design and integration of a filling proximal geometry, which provides superior congruency with the patient's cortical bone and prevents subsidence. Turning to FIGS. 1A-1D, FIG. 1A shows a side view of a femoral prosthetic implant designated generally at 10, illustrated with a medial side of the implant 10 facing downward. FIG. 1B is an isometric view of the implant 10. FIG. 1C is a perspective view of a femoral implant. FIG. 1D is a top view of an implant 10. The femoral prosthetic implant 10 comprises a first taper portion 12 which is configured for engagement with a substantially spherical femoral head (not illustrated). The head portion may be configured for articulating with an articulation surface, which articulation surface may be an acetabular cup or other surface used to assemble the socket portion of a ball and socket joint. The femoral head may be employed as the ball portion of a ball and socket joint. The first taper portion 12 comprises a proximal end 30 and a distal end 31. The proximal end 30 comprises a smooth surface that may engage a matching opening located within a head portion such that the head portion may be secured to the first taper portion. The distal end 31 is attached to a neck 14. The neck 14 has a proximal end 33 attached to the distal end 31 of the first taper portion 12 and a distal end 35. The tapered hip system 10 further comprises a stem portion 16 attached to the distal end 35 of the neck 14.

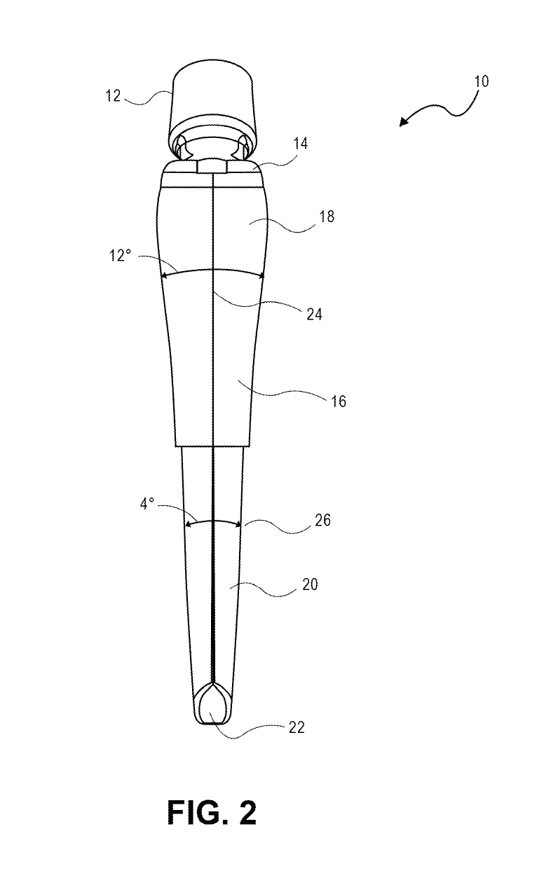

With less invasive approaches to total hip surgery, often the surgical window has a more restrictive geometry which can complicate insertion and placement of traditional length femoral components. Notably, the stem portion 16 of the implant 10 has significant advantages at least in part due to the reduced stem size and tapered profile. More particularly, the tapered profile provides ease of insertion, prevention of subsidence, and stability enhancement while allowing for a shortened femoral stem. The shape of the stem 16, having both a rounded portion and tapered profile, increases support during the initial implantation of the device when bone in-growth has not occurred. Despite the shorter stem as compared with many prior art devices, stability is achieved by promoting superior congruency with the cortical bone upon implantation such that micromotion and subsidence is decreased while promoting faster bone ingrowth. With specific reference to FIGS. 1A-1D, the stem portion 16 includes a proximal stem region 18 and a distal stem region 20. The proximal lateral shoulder of the device (as best illustrated in FIG. 1C) further facilitates the implantation of the stem in less invasive techniques and helps to avoid varus implant positioning. As will be described below with reference to FIG. 2, the stem portion 16 comprises a tapered design to prevent subsidence and promote ease of insertion of the implant 10. The proximal stem region 18 comprises a proximal anterior/posterior (AP) taper angle of 12 degrees. The distal stem region 20 includes a proximal anterior/posterior taper profile of 4 degrees. At the distal end of the implant 10, there is provided a distal tapered tip 22. The distal tapered tip 22 comprises a beveled construction which helps ease implantation of the device and protects the lateral cortical bone. The components of the implant 10 may be manufactured from titanium for cementless stem applications. However, it will be appreciated that the components could be manufactured from cobalt chrome molybdenum alloy for cemented stem applications for interfacing with cement and for providing reduced risk of fretting and corrosion at the stem neck junction. It should be noted that other material may be used that are presently known, or which may become known, in the art for manufacturing the above components, which can be readily determined by one of skill in the art.

Optionally, the exterior surface of the implant 10 may include a porous coating to aid in the apposition of bone to promote superior in-growth results. One such porous coating is the P² coating from DJO Surgical (Austin, Tex.). Advantageously, the coating is a titanium porous coating having small, three-dimensional beads. The porous material may be commercially pure titanium or CoCr alloy. Advantageously, the coating includes non-spherical beads to provide greater points of contact and a substantially rougher surface area to promote fixation and may include a “lava rock” appearance. More specifically, the coating can create instant micro-fractures in the trabeculae upon initial bite to the bone, which causes a relatively quick bone in-growth reaction. The pore sizes of the porous coating may be variable, including an inter-bead pore size of between about 200-525 microns and an intra-bead pore size of between about 25-65 microns. The average porosity of the coating is preferably greater than 50%. In a preferred embodiment, the average porosity is around 60%. Preferably, the porous coating provides a high surface roughness than spherical beads, thereby providing for greater bone apposition and percent bone in-growth than an uncoated implant.

By employing a metaphysial fill and biological fixation (i.e. bone growth into the implant), stability is greatly increased over prior art devices, allowing for solid long-term endurance of the disclosed implant. Moreover, in optimizing conforming proximal medial geometry, subsidence resistance is improved. Features of the tapered hip system 10 include a relatively short stem length as compared to prior art devices and a tapered shape to facilitate insertion and visualization as compared to other currently available tissue sparing designs. In one embodiment, the stem of the disclosed hip system is approximately 30 mm shorter than the stem of the Linear™ hip system. The hip system is constructed and shaped to avoid material interfering with insertion or version correction as well as to avoid cortical overlap at resection. A further feature of the tapered hip system includes the ability to engage substantially more proximal bone than prior art hip systems. The primary fixation of the tapered hip system is medial/lateral. Secondary fixation is anterior/posterior (hereinafter A/P) proximal while reducing cortical bone interference and allowing for version placement. Prior art devices such as the Linear™ hip stem include an uncemented stem having stability based on three point fixation, citation on fit and fill, and AML on distal fixation. The rectangular stem of the Linear™ device, however, does not conform with a patient's bone geometry. By contrast, the disclosed hip system includes fixation at the metaphysial/diaphysial junction, the region of the femur that has the strongest bone and is shaped like a funnel. This type of fixation approach capitalizes on the anatomy and physiology of this area inasmuch as fixation at this level provides excellent stability to subsidence, varus/valgus, and rotational forces akin to a cork in a bottle. The tapered design and proximal body shape of the hip system disclosed herein allows the bone to be loaded proximally, yielding positive bone remodeling and eliminating stress shielding as well as promoting diaphysial and metaphysial engagement.

With reference to FIG. 2, FIG. 2 is a lateral view of a prosthetic implant 10 to illustrate the tapered design, which allows for bone growth in the anterior/proximal direction at two different angles. The tapered elements of the implant prevent subsidence and ease insertion. In certain aspects, the tapered design facilitates congruency with the cortical bone. The tapered features of the hip stem system were designed to optimize proximal fill. The advantages of a tapered design include the prevention of subsidence and ease of insertion of the device, particularly with an anterior approach. The proximal stem region 18 comprises a second taper region 24 having a proximal anterior/posterior (AP) taper angle of approximately 12° (as measured as six degrees to middle) to enhance the stability of the device upon insertion. The 12 degree proximal taper thus provides inherent fixation by increasing surface area contact with the cortical bone structure which also promotes more physiological interface. At the distal stem region 20, there is a third taper region 26, whereby the stem tapers medially/laterally at an angle of approximately 4, the mismatch in the proximal 12° taper and the distal 4° taper provides a secondary means of fixation of the implant in the cortical bone. The mismatch could be greater or less than the preferred embodiment. Moreover, the third taper region 26 having a taper angle of less than the proximal body taper helps account for proximal/distal mismatch. The tapered configuration of the device further provides a substantially thinner lateral aspect (i.e. the portion of the proximal body opposite the side that mates to the cortical bone) as compared to prior art femoral prosthetic devices.

The relatively shorter stem of the hip system 10 described herein allows for ease of insertion as well as addresses proximal and distal mismatches. The average incision length of a standard total hip prosthesis is traditionally 22 cm long to accommodate the stems of prior art devices. The surgical window for less-invasive techniques such as an anterior approach insertion is generally between about 5-10 cm in length. Thus, a shorter stem allows for easier insertion into the bone without stressing the muscles and avoiding undue trauma to a patient as compared with prior art. Moreover, the relatively short stem eliminates distal modulus of elasticity mismatches that can cause thigh pain, helps to preserve the endosteal blood supply, and facilitates future revision surgery. The tapered design with short stem preserves bone and thus decreases the need to lateralize, which better accommodates the anterior approach. Finally, the tapered tip prevents diaphysial engagement but nevertheless allows the surgeon to fit the stem into the cavity.

The design of the hip stem is thus facilitated, in some embodiments, by a geometry optimization algorithm that provides detailed criteria to optimize stem geometry. The resulting hip system includes a highly conforming symmetric surface while minimizing the challenges of insertion. The overall length of the device is reduced to allow insertion through a smaller wound, and the conforming geometry is designed such that natural variations in bone geometry do not cause high stress contacts with the highly conforming surface

The proximal body shape was designed to optimize fit along the medial-anterior aspect of the proximal femur. To generate and evaluate the design, CAD files were virtually implanted into femur geometry represented by ADaMs standardized femur models for 5 sizes: mean, ±1 standard deviation from the mean, and ±2 standard deviations from the mean. The ADaMs models were constructed from a database of 73 male and female femur CT scans (mean age 67.5) taken from Northern Europe. Three of the individual femur scans used in the data base were selected for validating the fit of the proposed implant geometry.

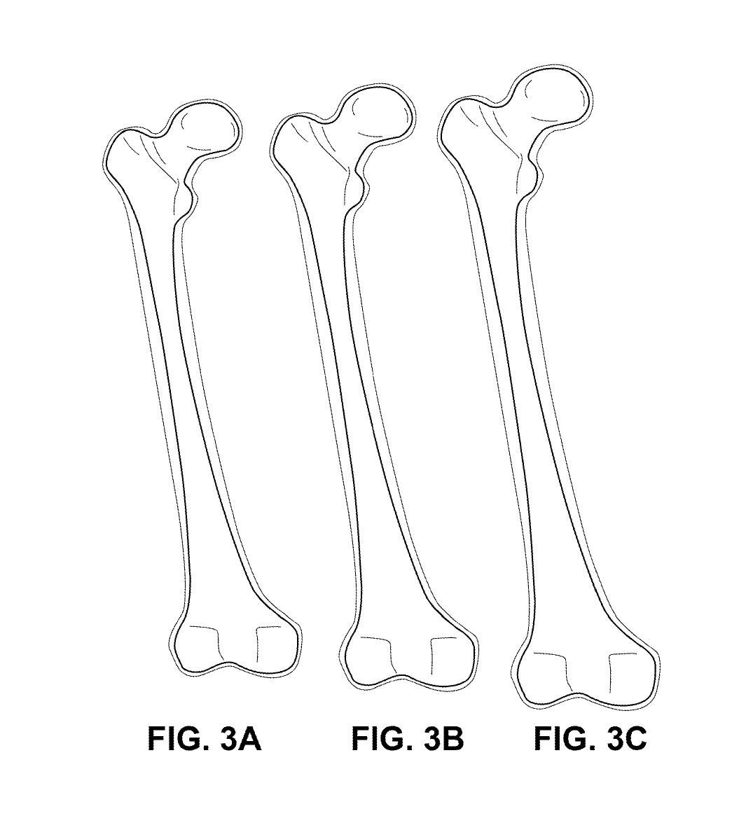

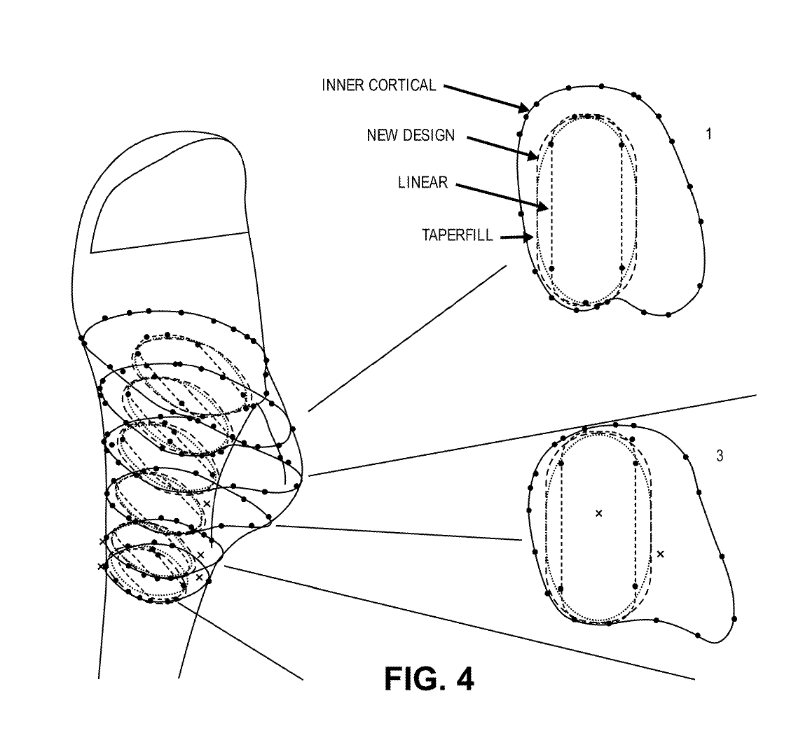

FIGS. 3A-3C illustrates ADaMs femur models. FIG. 3A illustrates −1 standard deviation, FIG. 3B illustrates the mean, and FIG. 3C illustrates +1 standard deviation. The fit of the design concepts was evaluated at standardized cross sections through the ADaMs models by measuring the length of congruent contact between the stem and the inner surface of the cortical shell. The distance was normalized to the distance measured for the Linear™ hip stem and the reported as a % change from the Linear™ hip stem. FIG. 4 is a model of the proximal region of a femur and illustrates the location of cross sections where implant and femur fit were evaluated. Bone models were sectioned between 5-10 mm increments. In the different sections, the implant shape was matched to the cortical shell geometry. Sketches were created at those sections of the shell and existing prior art implants and the A/P profile of the new design was derived according to the design parameters (wherein the cortical shell is the outermost boundary and the Linear™ implant is the innermost boundary).

An evaluation of the concept designs was also performed to evaluate the initial mechanical stability of the concept designs. The mean femur model was first virtually broached to form a cavity that was the negative of the proposed design concept. The implant geometry was fit into the cavity and the assembly transferred to Ansys where a frictionless contact analysis was defined. A single load vector corresponding to the peak load magnitude and direction during gait was used to load the implant neck. Deformation of the implant and cancellous bone cavity at a point medial and a point lateral were used to measure the relative implant-bone micromotion as an indication of implant stability. The results of this analysis are depicted below in Table 1.

TABLE 1
Comparison of Implant-Bone Micromotion between Linear ™ device,
a non-tapered device having a short stem, and the disclosed tapered device

| | Medial | Lateral |
|---|---|---|
| | Micromotion | Micromotion |
| | mm | mm |
| Linear ™ hip system | .501 | .571 |
| Fictitious | .542 | .627 |
| “short stem Linear” | | |
| Tapered hip system | .032 | .172 |

Due to variations in bone geometry with size, concepts built using the mean sized model were not always best fits for the other sizes, but by scaling the design concepts for the ±1, ±2 sizes, optimal shapes were developed to provide maximum congruency without interference across all sizes. To accommodate an anterior approach, a reduced length stem was desired, but it was recognized that simply cutting the stem on an existing implant could decrease initial stability thereby delaying or inhibiting bone growth.

Using the cross sectional plots, three dimensional designs were developed that had reduced overall length, were congruent with the cortical bone in the medial-anterior aspect of the femur, and consisted of a tapered design to minimize subsidence risk. The implant was designed to make use of increased proximal body congruency and tapered approach to maximize initial stability. Using the FEA model, micromotion between the implant and the broached bone was assessed for peak loading during the gait cycle. The clinically successfully Linear™ stem was used to benchmark excellent performance while a fictitious stem (Linear™ that was artificially shortened) was used as an indicator of minimal performance. The present implant, with its proximal conforming, tapered design had predicted micromotion less than one-third of Linear™.

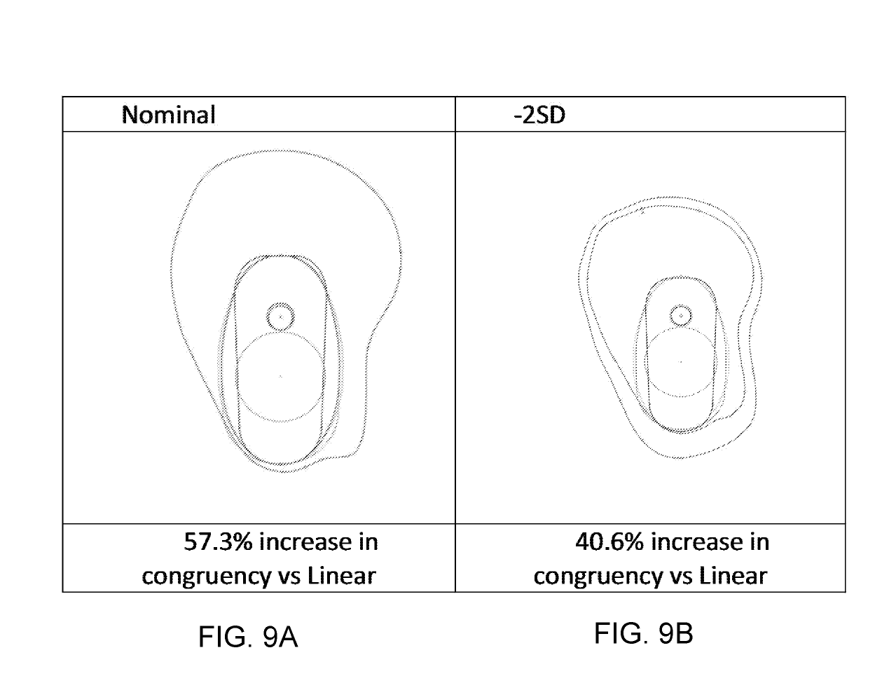

While a highly congruent implant shape was a design goal, the most highly congruent shape was not necessarily the optimal shape. A design that is highly congruent below the resection may present problems during insertion as the surgeon would be unable to visually confirm proper sizing and placement of the implant at the resection plane. The final hip stem design had a congruency that was 57.3% better than the Linear™ stem, easier to insert with an anterior approach, and provides visual feedback to the surgeon for sizing and placement as reflected in FIGS. 9A and 9B. FIG. 9A illustrates the congruency in the tapered design. FIG. 9B illustrates the congruency of the prior art Linear™ design. A comparison of the congruency between the tapered design in FIG. 9A to the prior art design of FIG. 9B shows that the present device thus comprises superior congruency with the patient's cortical bone.

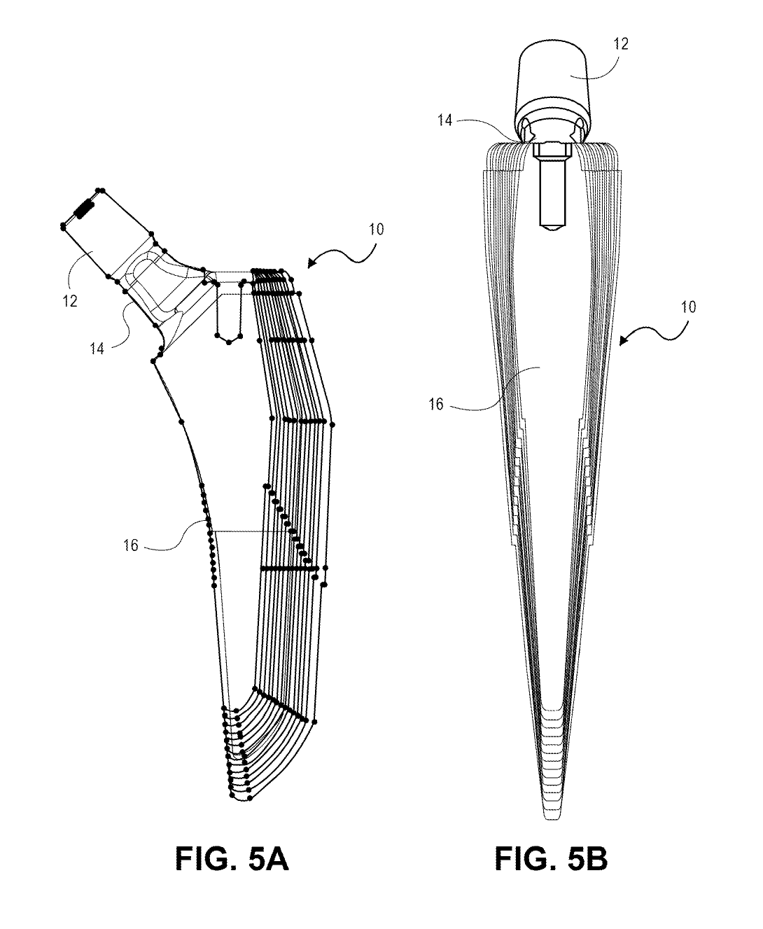

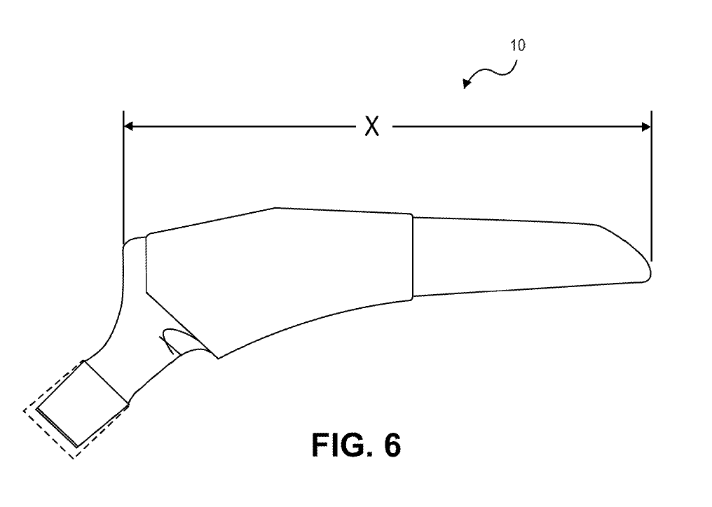

After optimizing fit and angles of the tapered design, the disclosed femoral prosthetic device provides even greater customization by offering numerous sizing options. In certain aspects, the disclosed hip system may include a plurality of both standard and lateralized offset stem sizes. The tapered design of the hip system provides immediate stable fixation with more physiologic loading proximally and promotes decreased risk of intraoperative complications, restoration of proper hip biomechanics and offset, intraoperative stability and expanded implant sizing options. As set forth with reference to FIGS. 5A and 5B, the sizing options both in standard and lateralized version of the stems enable a surgeon to select the best suited device to improve outcomes and success of the hip replacement system. FIG. 5A is a side view of the implant 10 illustrating the size progression of the stem region 16. FIG. 5B is a perspective view of the implant 10 having increasingly wider and longer stem region 16. Advantageously, the implant 10 can come in a plurality of sizes to best suit the patient anatomy. The stem region 16 is a relatively short stem as compared with many stems of femoral hip implants. As illustrated in FIG. 6, the stem region 16 has a length X that is between about 4 and about 5 inches from the proximal to distal end. The length X may vary according to the size of the implant. Advantageously, the implant may include a plurality of stem lengths suitable for use for a plurality of patient anatomies. Exemplary stem lengths X are set forth in Table 2.

TABLE 2
Sizing chart for stem lengths for tapered stem implant

| Stem Size | Length (inches) |
|---|---|
| 5 | 4.119 ± .020 |
| 6 | 4.179 ± .020 |
| 7 | 4.240 ± .020 |
| 8 | 4.300 ± .020 |
| 9 | 4.361 ± .020 |
| 10 | 4.421 ± .020 |
| 11 | 4.482 ± .020 |
| 12 | 4.543 ± .020 |
| 13 | 4.603 ± .020 |
| 14 | 4.664 ± .020 |
| 15 | 4.724 ± .020 |
| 16 | 4.785 ± .020 |
| 17 | 4.845 ± .020 |

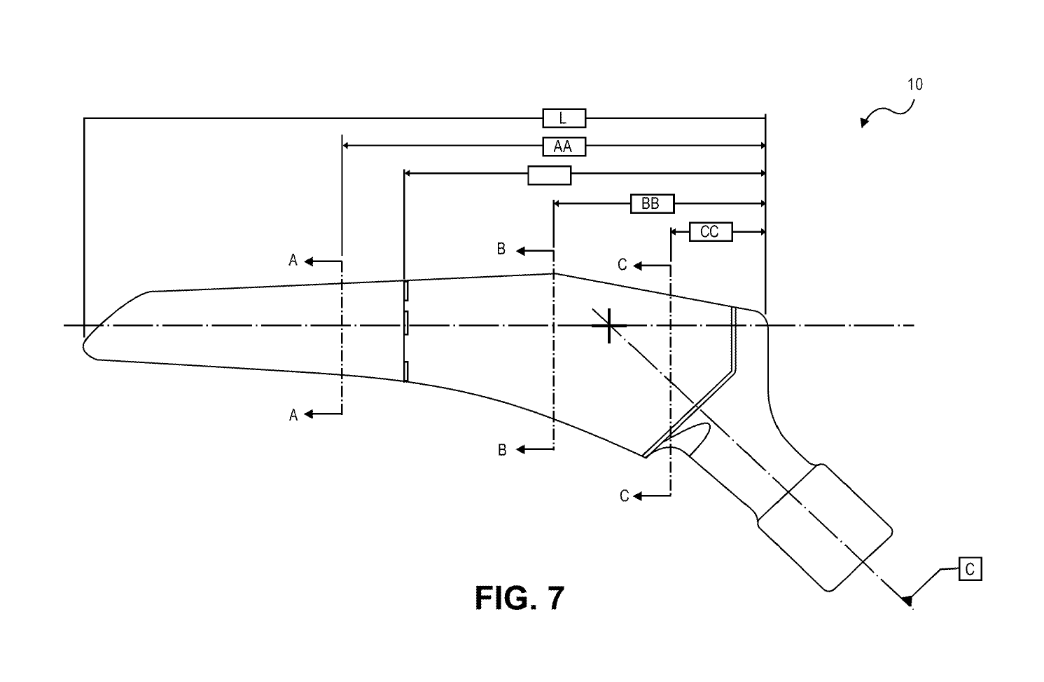

FIG. 7 is a side view of an implant 10 which when used in connection with the data set forth in Table 3, illustrates sizing and dimensional information relative to the implant 10. As illustrated, the stem region has a length L. The length of the stem from the third taper region to the proximal portion of the stem is measured as a length AA. The length from the second taper region to the proximal end of the stem is measured as a length BB. CC is the length of the device from the mid-section of the proximal region of the stem to the proximal end of the stem. Cross-sectional measurements of the device were also taken at the A-A axis, the B-B axis; and the C-C axis. The measurements of each labeled feature in FIG. 7 are reflected in the corresponding Table 3 below:

TABLE 3
Dimensions of the implant device (inches)

| Size | L | AA | BB | CC | A | B | C |
|---|---|---|---|---|---|---|---|
| 5 | | | | | | | |
| 6 | 4.179 | 2.756 | 1.376 | 0.641 | 0.599 | 0.532 | 0.927 |
| 7 | | | | | | | |
| 8 | | | | | | | |
| 9 | | | | | | | |
| 10 | 4.421 | 2.757 | 1.382 | 0.62 | 0.699 | 0.581 | 1.061 |
| 11 | | | | | | | |
| 12 | | | | | | | |
| 13 | | | | | | | |
| 14 | 4.664 | 2.757 | 1.386 | 0.641 | 0.795 | 0.622 | 1.202 |
| 15 | | | | | | | |
| 16 | | | | | | | |
| 17 | 4.845 | 2.84 | 1.388 | 0.641 | 0.675 | 0.675 | 1.304 |

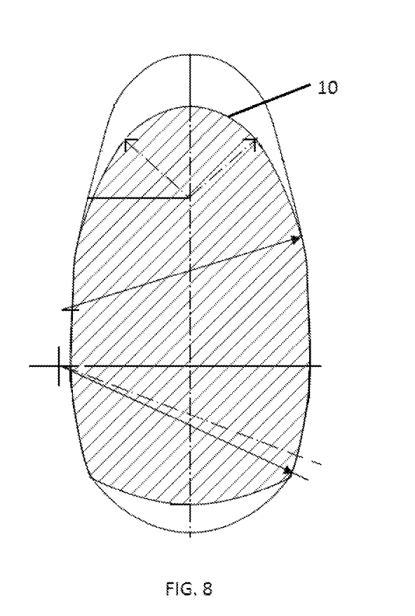

FIG. 8 is a cross-sectional view of a femoral implant device taken along the C-C axis (as illustrated in FIG. 7). FIG. 8 illustrates the congruency of the device when seated in a patient's bone. As illustrated, the device 10 is configured to provide a high level of congruency with the bone such that micromotion is dramatically reduced as compared with other femoral implant devices, despite the relatively short stem length. As illustrated, the widest portion A of the device fits snugly with a patient's bone. The cross-sectional perimeter can include both rectilinear and/or curvilinear portions which may be used to appropriately seat the implant device in the femur. For example, from the top of FIG. 8 and moving in a clockwise direction, the cross-sectional perimeter includes two regions of successively increasing radius of curvature, approaching a rectilinear portion of the cross-section in the region of 3 o'clock, and then gradually decreasing again till approximately 4:30, when the perimeter shape takes a sharp turn inward. Between 4:30 and 6 o'clock, the shape of the cross-sectional perimeter maintains a relatively shallow curvature. The shape of the right half of the cross-sectional perimeter may then be mirrored through the left half of the cross-section. The particular arc lengths for each section, as well as the particular radii of curvature (if any) may be selected to suit the size of the bone in which the device will be implanted. By including portions with differing radii of curvature and/or rectilinear portions in the cross-sectional shape, the implant according to embodiments may be easily seated within the cavity in which it is implanted, and the appropriate seating may be more readily visualized by the surgeon.

A method of treating a patient suffering from a hip disability is likewise contemplated. As used herein, the term “disability” includes without limitation non-inflammatory degenerative joint disease such as osteoarthritis and avascular necrosis of the natural femoral head, rheumatoid arthritis, correction of functional deformity, revision procedures where other treatments or devices have failed, treatment of non-union, and hip fractures. Utilizing the embodiments disclosed herein, a hip stem facilitates insertion than longer stem such as the Linear™ stem. The disclosed embodiment further provides increased fixation stability due to the proximal body conformity and a reduced chance of initial subsidence by providing tapered features. The inventive embodiments further provide for lower fracture risk, relative to highly conforming implants without regard to anatomical variation because of the intentional biasing of conformity to be maximum and consideration of the resection level where surgeon gets visual feedback of stem fit.

It will be appreciated that one of the features of the disclosed prosthetic device is its tapered profile and shorter stem. The configuration is particularly well suited for providing enhanced congruency with the cortical bone of the patient while allowing for visual feedback to the surgeon at the resection plane when the implant is inserted. The combination of a highly congruent device with a short stem allows for increased support during implantation as well as facilitating insertion, despite the congruency between the device and cortical bone. Enabling visual feedback to the surgeon at the resection plan prevents weakening of the patient bone caused by the surgeon trying to force the implant position beyond contact with the cortical shell. While the implant can be used with any number of incision approaches, it is particularly well-suited for use in an anterior approach because of the reduced incision length of such an approach coupled with the shorter stem of the disclosed device and opportunity for visual feedback for the surgeon at the resection plane.

An improved method for treating osteoarthritis and fractures of a hip of a patient through replacement of the hip with a femoral hip stem that facilitates insertion of the stem and visual confirmation of fit to patient's anatomy while also reducing trauma to tissue and femur of the patient is provided. The method includes providing a femoral hip implant as disclosed herein. The femoral hip implant is dimensioned such that as dimensions of the femur increase in both an anterior and posterior dimension from a diaphysial region of the femur to a proximal metaphysial region of the femur, dimensions of the proximal end of the stem also increase in both an anterior and posterior dimension relative to a distal end of the stem to provide a stem shape congruent to the femur of the patient and optimize proximal geometry of the stem for insertion of the stem. The tapered design of the implant includes at the distal stem region, a tapering of the stem medially/laterally at an angle of approximately 4°. As detailed above, this reduced distal taper avoids stress shielding common with prior art devices. Moreover, the 4 degree distal taper helps account for proximal/distal mismatch. The proximal stem region 18 comprises a proximal anterior/posterior (AP) taper angle of approximately 12° to enhance the stability of the device upon insertion. The distal tip is beveled and likewise angled to secure the device in the cavity of the femur.

The method further includes making an incision in the patient. The incision is advantageously less than the standard hip replacement surgery of between about 20-22 cm. In some aspects, the incision is instead between about 5 and 10 cm. Once an incision has been made, a portion of the femur is removed to create a cavity. The stem of the implant is positioned in the cavity, wherein the proximal geometry of the stem is visible at a resection plane of the stem to allow visual feedback of the positioning of the stem.

The implant as described herein is useful in hip replacement surgery whether the incision is a posterior, anterior, or lateral approach. In certain aspects, the implant is particularly well suited for an anterior approach to hip replacement.

In another aspect, an improved method for treating osteoarthritis and fractures of a hip of a patient through replacement of the hip with a femoral hip stem that minimizes subsidence risk, reduces trauma to tissue and femur of the patient, and improves recovery time is provided. The method includes providing a femoral hip stem wherein the stem has both a distal taper and a proximal taper, the distal taper minimizing stress shielding and the proximal taper increasing fixation and rotational stability through increased surface area contact with cortical bone structures. The method further includes making a 5-10 cm incision in a patient, removing a portion of the femur to create a cavity, and setting the stem in the cavity, wherein the proximal body shape and the distal and proximal taper limit micromotion between the stem and the femur in both the medial and lateral dimensions. The incision may be an anterior incision that reduces soft tissue damage. Optionally, the surface of the stem of the implant is coated with a porous coating to facilitate rapid bone in-growth. The tapers of the femoral implant include the distal taper of about 4 degrees and a proximal taper of about 12 degrees.

A kit for an improved treatment of osteoarthritis through replacement of a hip is likewise contemplated. The kit may include a femoral hip stem wherein as dimensions of a femur increase in both an anterior and posterior dimension from a diaphysial region of the femur to a proximal metaphysial region of the femur, dimensions of a proximal end of the stem also increase in both an anterior and posterior dimension relative to a distal end of the stem to provide a stem shape congruent to the femur and optimize proximal geometry of the stem for insertion of the stem; and instructions for positioning and setting the femoral hip stem in a cavity of the femur through an anterior incision; wherein the instructions include a guide to visually confirm correct positioning and setting of the femoral hip stem.

The above description of disclosed embodiments is provided to enable any person skilled in the art to make or use the invention. Various modifications to the embodiments will be readily apparent to those skilled in the art; the generic principles defined herein can be applied to other embodiments without departing from spirit or scope of the invention. Thus, the invention is not intended to be limited to the embodiments shown herein but is to be accorded the widest scope consistent with the principles and novel features disclosed herein.

What is claimed is:

1. A tapered femoral prosthetic implant for use in total hip replacement, comprising:
a first taper portion configured to receive and attach to a femoral head element; and
a stem portion, wherein said stem portion comprises a proximal stem region and a distal stem region, wherein the proximal stem region comprises a 12 degree anterior proximal taper; and wherein the distal stem region comprises a 4 degree distal taper.

2. The implant of claim 1, wherein the proximal stem region comprises medial wall, wherein the medial wall is shaped to substantially conform to an inner medial wall of a femur canal when the stem portion is fully seated.

3. The implant of claim 1, wherein the stem portion has a length of between about 4 and about 5 inches.

4. The implant of claim 1, wherein said implant has an exterior surface, and wherein said exterior surface comprises a porous coating.

5. The implant of claim 1, wherein said stem portion further comprises a distal tip, wherein said distal tip is beveled.

6. The implant of claim 5, wherein said distal tip is configured to facilitate insertion of said implant during implantation.

7. An improved method for treating osteoarthritis and fractures of a hip of a patient through replacement of the hip with a femoral hip stem that facilitates insertion of the stem and visual confirmation of fit of the stem while also reducing trauma to tissue and femur of the patient, the method comprising:
providing the femoral hip stem wherein as dimensions of the femur increase in both an anterior and posterior dimension from a diaphysial region of the femur to a proximal metaphysial region of the femur, dimensions of a proximal end of the stem also increase in both an anterior and posterior dimension relative to a distal end of the stem to provide a stem shape congruent to the femur of the patient and optimize proximal geometry of the stem for insertion of the stem;
making an incision in the patient;
removing a portion of the femur to create a cavity;
positioning the stem in the cavity, wherein the proximal geometry of the stem is visible at a resection plane of the stem to allow visual feedback of the fit of the stem.

8. The method of claim 7, wherein the stem shape is most congruent to the femur of the patient at the resection plane of the stem.

9. The method of claim 7, wherein the distal end of the stem includes a beveled tip to facilitate insertion of the implant in a patient.

10. The method of claim 7, wherein the proximal end of the stem includes a plurality of contact points at the resection plane to allow visual feedback that the plurality of contact points are congruent with the patient anatomy.

11. The method of claim 7, wherein the dimensions of stem decrease in both a medial and a lateral dimension from the proximal end of the stem to the distal end of the stem.

12. The method of claim 7, wherein the dimension of the stem decreases in a distal dimension from the proximal end of the stem to the distal end of the stem.

13. The method of claim 7, wherein the incision is between 5 and 10 cm.

14. The method of claim 7, wherein the incision is one of a posterior, anterior, or lateral incision.

15. An improved method for treating osteoarthritis and fractures of a hip of a patient through replacement of the hip with a femoral hip stem that minimizes subsidence risk, reduces trauma to tissue and femur of the patient, and improves recovery time, the method comprising:
providing a femoral hip stem, wherein the stem has both a distal taper and a proximal taper, said distal taper configured to reduce stress shielding and said proximal taper configured to increase fixation and rotational stability through increased surface area contact with cortical bone structures;
making an incision in the patient;
removing a portion of the femur to create a cavity;
setting the stem in the cavity, wherein the distal and proximal tapers limit micromotion between the stem and the femur in both a medial and lateral dimension; and
obtaining visual feedback at a resection plane to confirm implantation

16. The method of claim 15, wherein the incision is an anterior incision that reduces soft tissue damage.

17. The method of claim 15, wherein a surface of the stem is coated with a porous coating to facilitate rapid bone in-growth.

18. The method of claim 15, wherein the distal taper is a 4 degree distal taper.

19. The method of claim 15, wherein the proximal taper is a 12 degree proximal taper.

20. The method of claim 15, wherein said stem has a length of between about 4 and about 5 inches.

21. A kit for an improved treatment of osteoarthritis through replacement of a hip, the kit comprising:
a femoral hip stem wherein as dimensions of a femur increase in both an anterior and posterior dimension from a diaphysial region of the femur to a proximal metaphysial region of the femur, dimensions of a proximal end of the stem also increase in both an anterior and posterior dimension relative to a distal end of the stem to provide a stem shape congruent to the femur and optimize proximal geometry of the stem for insertion of the stem; and
instructions for positioning and setting the femoral hip stem in a cavity of the femur through an anterior incision.

22. The kit of claim 21, wherein said femoral hip stem comprises a porous coating.

23. The kit of claim 21, wherein said instructions comprise a guide to visually confirm correct positioning and setting of the femoral hip stem.

24. The kit of claim 21, wherein the stem has a length of between about 4 and about 5 inches.

25. The kit of claim 21, wherein the stem comprises a proximal stem region and a distal stem region, wherein the proximal stem region comprises a 12 degree anterior proximal taper; and wherein the distal stem region comprises a 4 degree distal taper.

26. A femoral hip prosthetic device, comprising:
a first taper portion configured to engage a femoral head; and
a femoral stem; said stem comprising:
a second taper portion having a 12 degree anterior proximal taper;
a third taper portion having a 4 degree distal taper; and
a beveled distal tip;
wherein said stem has a length of between about 4 to about 5 inches.

27. The prosthetic device of claim 26, further comprising an exterior porous coating.
